(19) United States
(12) Patent Application Publication
(10) Pub. No.: US 2022/0296518 A1
(43) Pub. Date: Sep. 22, 2022

(54) LIPID-BASED NANOPARTICLE DELIVERY SYSTEM FOR HYDROPHILIC CHARGED COMPOUND

(71) Applicant: National Taiwan University, Taipei (TW)

(72) Inventors: Chin-tin Chen, Taipei (TW); Chia Ying Lee, New Taipei City (TW); Tsuimin Tsai, Taipei (TW); Po-Chun Peng, Taipei (TW)

(21) Appl. No.: 17/697,941

(22) Filed: Mar. 18, 2022

Related U.S. Application Data
(60) Provisional application No. 63/163,005, filed on Mar. 18, 2021.

(51) Int. Cl. A61K 9/127; A61P 35/00

(52) U.S. Cl. CPC A61K 9/1272; A61K 9/1277; A61P 35/00

(57) ABSTRACT

A lipid-based nanoparticle (LNP) with high DL ratio and normalized release. The LNP of the present invention comprises an outer lipid monolayer encapsulating a plurality of lipid-active pharmaceutical ingredient (API) complexes, wherein each lipid-API complex comprises a complex of anionic lipid and API wherein the API comprises a positively charged form of an API and wherein the outer lipid monolayer of the LNP comprises neutral lipids. The present invention further comprises a method of preparation of the LNP of the present invention.

### CROSS REFERENCE TO RELATED APPLICATIONS

The present application claims priority to U.S. Provisional Application No. 63/163,005, filed on Mar. 18, 2021, entitled “Lipid Based Nanoparticle Delivery System for Hydrophilic Charged Compound.”

### FIELD OF THE INVENTION

The present invention relates to lipid-based nanoparticles (LNP) with high drug to lipid (D/L) ratio and normalized release, method of preparation of the LNP and uses thereof.

### BACKGROUND OF THE INVENTION

Lipid-based nanoparticles (LNP) have become important vehicles for delivery of various therapeutic compounds to targeted sites since LNPs provide advantages over other drug delivery means due to better biodistribution and toxicity mitigation. Specifically, the small size of LNPs better avoids removal from the bloodstream by the reticuloendothelial system so that they can circulate for a longer period of time in a patient. In addition, LNPs are able to better target diseased sites such as cancer tumors and inflammation sites by extravasation through leaky vasculature of the cancer tumor and disease inflamed regions.

However, to provide even more effective therapy using LNPs, there is a need to substantially increase the drug to lipid ratio (D/L ratio) of LNPs so that more of the therapeutic compounds can be carried per LNP. In addition, there is a need to improve the ability of the LNP to target disease site by increasing release rate at the targeted disease site while maintaining stability elsewhere.

The use of rapid mixing to increase the effect of electrostatic forces between components comprising the LNP result in substantially higher D/L ratio as well as increased capability for targeting diseased sites than prior art LNP. Specifically, the use of rapid mixing to more effectively mix negatively electrostatically charged lipids with positively electrostatically charged API can substantially increase D/L ratio. Furthermore, the typically net negative electrostatic charge environment of acidic cancer tumor regions helps to increase the release rate of the LNP at the target disease site by disrupting the structure of LNP that comprises charged components such as charged lipids and active pharmaceutical ingredients.

An objective of the present invention comprises substantially raising drug to lipid ratio of the LNP of the present invention compared to those of existing LNPs. Another objective of the invention comprises improving ability of the LNP of the present invention to target tumors thus improving therapeutic effects thereof.

### BRIEF DESCRIPTION OF THE DRAWINGS

FIG. 1 FIG. 1A illustrates prior art LNP while FIG. 1B illustrates LNP 1 of the present invention.

FIG. 2 illustrates a method of preparation 100 for the LNP 1 of the present invention.

FIG. 3 illustrates various factors that can affect properties of the LNP 1 of the present invention.

FIG. 4 illustrates properties of embodiments of the LNP 1 of the present invention encapsulating cisplatin, doxycycline or doxorubicin.

FIG. 5 illustrates the process by which LNP 1 of the present invention may enter a tumor cell and release encapsulated API 30.

FIG. 6 illustrates in vitro release of dox of LNP 1 of the present invention in pH 7.4 and pH 5.4 environments at physiological temperature of about 37° C.

FIGS. 7A and 7B illustrate in vivo therapeutic efficacy of LNP 1 of the present invention encapsulating doxorubicin against tumor growth (FIG. 7A) and survival (FIG. 7B) of mice.

FIG. 8 illustrates in vitro Staphylococcus aureus antibacterial susceptibility to LNP 1 of the present invention encapsulating doxycycline as compared to free form doxycycline both at MIC 0.4 mcg/ml.

FIG. 9 illustrates in vitro methicillin-resistant Staphylococcus aureus antibacterial susceptibility to LNP 1 of the present invention encapsulating doxycycline as compared to free form doxycycline both at MIC 0.4 mcg/ml.

FIG. 10 illustrates in vitro toxicity to NIH-3T3 cell line of LNP 1 of the present invention encapsulating doxycycline as compared to toxicity of free form doxycycline at various concentrations of treatment for 1 hour at physiological temperature of about 37° C.

FIG. 11 illustrates an exemplary embodiment of a rapid mixing system 200.

FIG. 12 illustrates an exemplary embodiment of microfluidic device 300 for performing rapid mixing.

### DETAILED DESCRIPTION OF THE PRESENT INVENTION

The compositions of the present invention can comprise, consist of, or consist essentially of the essential elements and limitations of the invention described herein, as well as any of the additional or optional ingredients, components, or limitations described herein.

As used in the specification and claims, the singular form “a” “an” and “the” include plural references unless the context clearly dictates otherwise. For example, the term “a” cell includes a plurality of cells, including mixtures thereof.

“About” in the context of amount values refers to an average deviation of maximum ±20%, preferably ±10% or more preferably ±5% based on the indicated value. For example, an amount of about 30 mol % anionic lipid refers to 30 mol %±6 mol %, preferably 30 mol %±3 mol % or more preferably 30 mol %±1.5 mol % anionic lipid with respect to the total lipid/amphiphile molarity.

“Lipid” refers to its conventional sense as a generic term encompassing fats, lipids, and alcohol-ether soluble constituents of protoplasm, which are insoluble in water. Lipids are composed of fats, fatty oils, essential oils, waxes, steroid, sterols, phospholipids, glycolipids, sulfolipids, aminolipids, chromolipids, and fatty acids. The term encompasses both naturally occurring and synthetic lipids. Preferred lipids in connection with the present invention are: steroids and sterol, particularly cholesterol, phospholipids, including phosphatidyl and phosphatidylcholines and phosphatidylethanolamines, and sphingomyelins. Where there are fatty acids, they could be about 12-24 carbon chains in length, containing up to 6 double bonds. The fatty acids are linked to the backbone, which may be derived from glycerol. The fatty acids within one lipid can be different (asymmetric), or there may be only 1 fatty acid chain present, e.g., lysolecithins. Mixed formulations are also possible, particularly when the non-cationic lipids are derived from natural sources, such as lecithins (phosphatidylcholines) purified from egg yolk, bovine heart, brain, or liver, or soybean.

“Neutral lipids” are lipids which have a neutral net charge. “Anionic lipids” are lipid molecules which have a negative net charge. These can be selected from sterols or lipids such as cholesterol, phospholipids, lysolipids, lysophospholipids, sphingolipids or pegylated lipids with a negative net charge. “Amphiphilic lipids” are lipid molecules which exhibit both hydrophilic and hydrophobic properties. These can be selected from sterols or lipids such as cholesterol, phospholipids, lysolipids, lysophospholipids, sphingolipids or pegylated lipids with amphiphilic properties, which may have a negative, neutral, or positive net charge. Useful neutral and anionic lipids thereby include: phosphatidylserines, phosphatidylglycerols, phosphatidylinositols (not limited to a specific sugar), fatty acids, sterols containing a carboxylic acid group for example, cholesterol, phosphatidylethanolamines (PE) such as 1,2-diacyl-sn-glycero-3-phosphoethanolamines including, but not limited to 1,2-dioleoylphosphoethanolamine (DOPE), 1,2-distearoylphosphoethanolamine (DSPE), or 1,2-dihexadecoylphosphoethanolamine (DHPE), phosphatidylcholines (PC) such as 1,2-diacyl-glycero-3-phosphocholines including, but not limited to 1,2-distearoylphosphocholine (DSPC), 1,2-dipalmitoylphosphocholine (DPPC), 1,2-dimyristoylphosphocholine (DMPC), egg PC or soybean PC and sphingomyelins. The fatty acids linked to the glycerol backbone are not limited to a specific length or number of double bonds. Phospholipids may also have two different fatty acids.

An “effective amount” is an amount sufficient to effect beneficial or desired results. An effective amount can be administered in one or more administrations, applications or dosages.

A “subject,” “individual” or “patient” is used interchangeably herein, which refers to a vertebrate, preferably a mammal, more preferably a human. Mammals include, but are not limited to, murines, simians, humans, farm animals, sport animals, and pets.

The “polydispersity index” (PDI) is defined as a measure of the heterogeneity of sizes of molecules or particles in a mixture such as a mixture of LNPs calculated as square of the standard deviation of the size distribution divided by the square of the mean of the size distribution.

The “drug to lipid ratio” (D/L ratio) is defined as the weight of drug (mg) divided by the weight of phospholipid (mg) in the lipid nanoparticle.

The “normalized release” is defined as release ratio in pH 5.4 environment divided by release ratio in pH 7.4 environment wherein the release ratio is defined as the percentage of API released from LNP. A high normalized release is a result of high release ratio in pH 5.4 environment as compared to the release ratio in pH 7.4 environment. Since tumor sites present acidic environment, a high normalized release is a metric indicating tumor targeting capability of an LNP.

The “rapid mixing” as defined by Djamel Ghernaout and Heinrich Roder articles^{1,2}, hereby incorporated in their entirety, is mixing that provides quick and efficient dispersion of two or more fluids to achieve complete mixing of the two or more fluids including mixing of various components comprising each fluid. Various types of rapid mixing have been developed. These include mechanical mixing, which is conventionally used for coagulant mixing in water treatment, diffusion mixing by a pressured water jet, in-line static mixing, in-line mechanical mixing, hydraulic mixing and mechanical flash mixing. ¹ Djamel Ghernaout and Ahmed Boucherit (2015), Journal of Research & Developments in Chemistry, DOI: 10.5171/2015.926518, page. 22.² Heinrich Order, Kosuke Make, Hong Cheng and M. C. Ramachandra Shastry, Methods 34(2004) 15-27

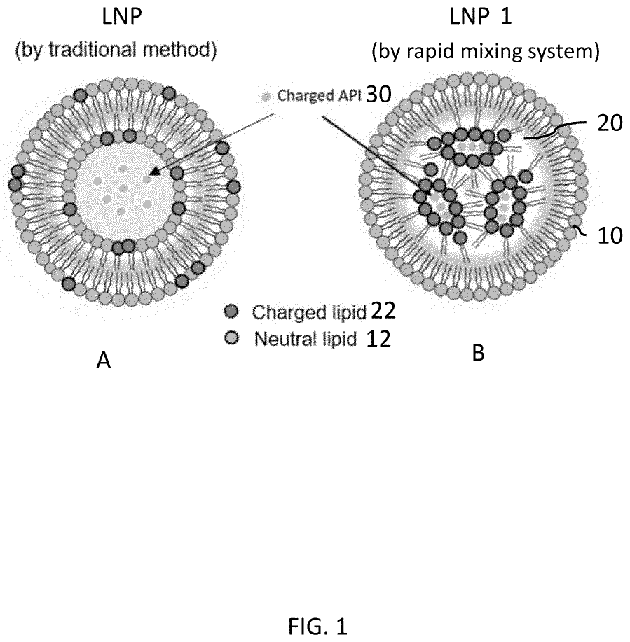

FIG. 1B illustrates an embodiment of the LNP 1 of the present invention while FIG. 1A illustrates prior art LNP. Compared to prior art LNP, the LNP 1 of the present invention provides substantially higher D/L ratio as well as normalized release as discussed further below in connection with the Examples.

As illustrated in FIG. 1B, an embodiment of the LNP 1 of the present invention comprises a lipid monolayer 10 encapsulating a plurality of lipid-API complexes 20 wherein each of the lipid-API complexes 20 comprises a complex of anionic lipids 22 and positively charged active pharmaceutical ingredient (API) 30.

In an embodiment, the lipid monolayer 10 of the LNP 1 of the present invention preferably comprises neutral lipids 12 such as but not limited to L-α-phosphatidylcholine, hydrogenated soybean phosphatidylcholine (HSPC), 1,2-dioeoyl-sn-glycero-3-phosphocholine (DOPC), 1,2-dioleoylphosphoethanolamine (DOPE), 1,2-distearoylphosphoethanolamine (DSPE), and/or polyethyleneglycol-derivated distearoylphosphatidylethanolamine (PEG-DSPE), or 1,2-dihexadecoylphosphoethanolamine (DHPE), phosphatidylcholine (PC) such as 1,2-diacyl-glycero-3-phosphocholines including but not limited to 1,2-distearoylphosphocholine (DSPC), 1,2-dipalmitoylphosphocholine (DPPC), and 1,2-dimyristoyl-sn-glycero-3-phosphocholine (DMPC). In an embodiment, the lipid monolayer 10 of the LNP 1 of the present invention preferably comprises less than 50%, 20%, 15%, 10%, 5% or 1% of positively or negatively charged lipids.

In an embodiment, anionic lipids 22 may comprise phosphatidylglycerol, cardioplipin, diacylphosphatidylserine, diacylphosphatidic acid, lysylphosphatidylglycerol, egg L-α-phosphatidylglycerol (EPG), 1,2-dimyristoyl-sn-glycero-3-phosphoglycerol (DMPG), 1,2-dipalmitoyl-sn-glycero-3-phosphoglycerol (DPPG), 1,2-dioleoyl-sn-glycero-3-phospho-(1′-rac-glycerol) (DOPG), 1,2-dioeoyl-sn-glycero-3-phosphate (DOPA), 1,2-dioleoyl-sn-glycero-3-phospho-L-serine (DOPS), and other anionic modifying groups joined to neutral lipids such as HSPC. In another embodiment, the anionic lipid 22 does not comprise any phosphatidyl glycerol or its derivatives. In an embodiment, the LNP 1 of the present invention does not comprise any phosphatidyl glycerol or its derivatives. The charge on each anionic 22 lipid may be in the range of about −1 to −5 eV, about −1 to −3 eV, about −1 to −2 eV. In an embodiment, the lipid content of the lipid-API complexes 20 preferably each comprises less than 50%, 20%, 15%, 10%, 5% or 1% of neutral lipids. In an embodiment, the lipid-API complex 20 comprises the anionic lipid 22 surrounding the API 30. In an embodiment, the lipid-API complex 20 comprises the anionic lipid 22 encapsulating the API 30. In an embodiment, the lipid-API complex 20 comprises a micelle comprising a lipid monolayer encapsulating the API 30 wherein the lipid monolayer comprises the anionic lipid 22.

The lipid monolayer 10 of the LNP 1 of the present invention may further comprise neutral lipids with PEG such as DSPE-PEG2000 and/or DMG-PEG. In an embodiment, the PEG comprises in the range of about 1% to about 10% by weight of the neutral lipids. The LNP 1 and the lipid-API complexes 20 may each further comprise steroids such as cholesterol, cholestanol, lanosterol, and the like. In an embodiment, the lipid monolayer 10 of the LNP 1 comprises more than 60%, 80%, 90%, or 95% neutral lipids. In another embodiment, the lipid content of the lipid-API complex 20 comprises more than 60%, 80%, 90%, or 95% anionic lipids 22.

In an embodiment, the API 30 comprises a positively charged and hydrophilic compound. In another embodiment, the API 30 comprises a positively charged and hydrophilic antineoplastic drug. In another embodiment, the API 30 comprises positively charged forms of doxorubicin (dox), cisplatin (cDDP) or doxycycline (doxy). In an embodiment, the API 30 obtains the positive charge by hydration process. In an embodiment, doxorubicin, cisplatin, or doxycycline obtains positive charge by means of hydration as known in the art. In an embodiment, cisplatin may be hydrated for about 10 min to about 180 min, about 20 min to about 150 min, about 30 min to about 120 min, about 40 min to about 90 min, and about 50 min to about 70 min. In an embodiment, doxorubicin or doxycycline may be hydrated for about 30 sec to about 50 min, about 1 min to about 30 min, about 5 min to about 20 min, or about 7 min to about 15 min. In another embodiment, the lipid-API complexes 20 may further encapsulate adjuvants, or one or more additional APIs, etc. . . .

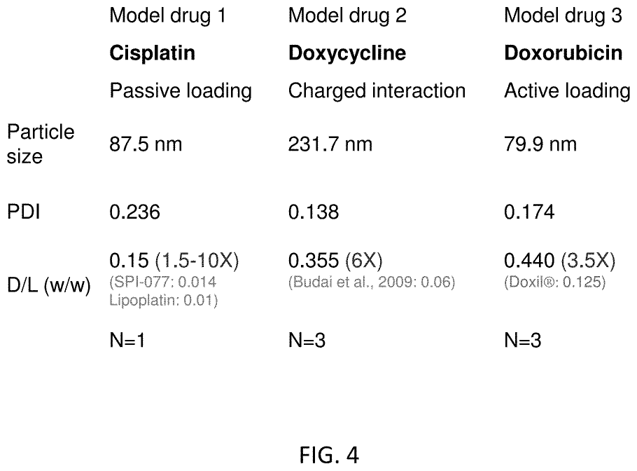

As illustrated in FIG. 4, the drug to lipid ratio for prior art LNP encapsulating cisplatin is from about 0.014 to about 0.1.³ In contrast, using the formulation and method of preparation disclosed in the Examples below, the drug to lipid ratio of the LNP 1 encapsulating cisplatin of the present invention is substantially higher at from about 0.12 to about 0.2 such as about 0.12, about 0.13, about 0.14 or about 0.15, about 0.16, about 0.17, about 0.18, about 0.19 or about 0.2 including all numbers ranges and numbers falling within these values. In addition, the drug to lipid ratio for prior art LNP encapsulating doxy is about 0.06.⁴ In contrast, again using the formulation and method of preparation disclosed in the Examples below, the drug to lipid ratio of the LNP 1 encapsulating doxy of the present invention is substantially higher from about 0.16 to about 0.45 such as about 0.16, about 0.2, about 0.25, about 0.3, about 0.35, about 0.4 or about 0.45 including all numbers ranges and numbers falling within these values. In addition, the drug to lipid ratio for prior art LNP 1 encapsulating dox of the present invention is about 0.125.⁵ In contrast, using the formulation and method of preparation disclosed in the Examples below, the drug to lipid ratio of the LNP 1 encapsulating dox of the present invention is substantially higher at about 0.15 to about 0.55 such as about 0.1, about 0.15, about 0.2, about 0.25, about 0.3, about 0.35, about 0.4, about 0.45, about 0.5 or about 0.550 including all numbers ranges and numbers falling within these values. ³ http://www.hoajonline.comhournals/pdf/2052-9341-4-2.pdf⁴ Budai, M., et al., Liposomal oxytetracycline and doxycycline: studies on enhancement of encapsulation efficiency. Drug Discov Ther, 2009. 3(1): p. 13-7.⁵ Nordström, Rickard et al. “Quantitative Cryo-TEM Reveals New Structural Details of Doxil-Like PEGylated Liposomal Doxorubicin Formulation.” Pharmaceutics vol. 13, 1 123. 19 Jan. 2021, doi:10.3390/pharmaceutics13010123

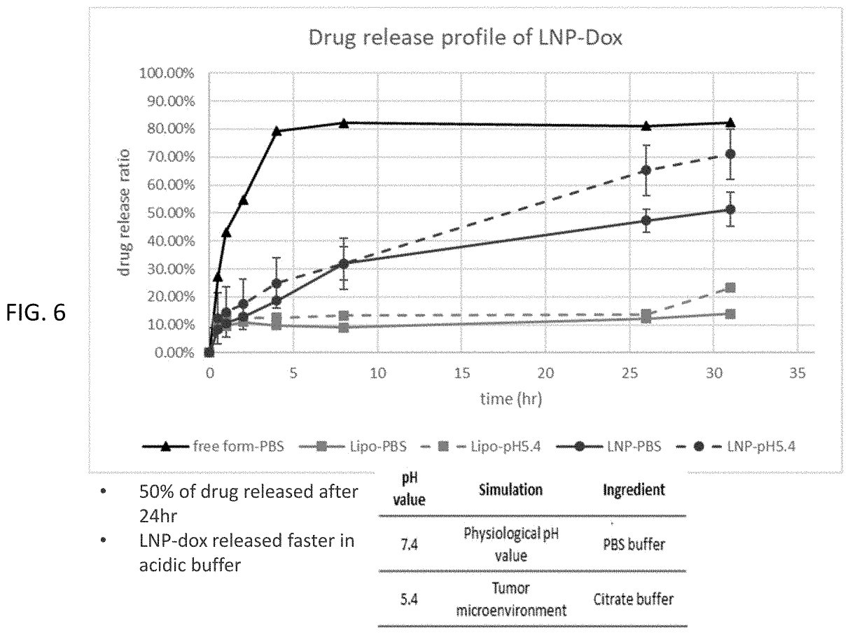

In an embodiment, the LNP 1 of the present invention possesses substantially higher drug to lipid ratio (D/L ratio) as well as substantially higher normalized release compared to the prior art. As discussed above, high normalized release indicates substantially higher release ratio within acidic environment than within neutral environments. Since cancer tumor tissues presents an acidic environment, a high normalized release is a metric that indicates the ability of LNP to target cancer cells as discussed in detail below in connection with the Examples and FIG. 6.

In an embodiment, the LNP 1 of the present invention does not comprise a therapeutically effective amount of nucleic acids, nucleotides or polynucleotides including RNA or RNA-based or RNA derived API such as siRNA, micro RNA, antisense oligonucleotides, ribozymes, plasmids and/or immune stimulating nucleic acids. In another embodiment, the LNP 1 of the present invention comprises less than 5%, 2%, 1%, 0.1% or 0.001% by molar ratio of nucleic acids, nucleotides or polynucleotides including RNA or RNA-based or RNA derived API siRNA, micro RNA, antisense oligonucleotides, ribozymes, plasmids and/or immune stimulating nucleic acids. In another embodiment, the LNP 1 of the present invention is completely free of nucleic acids, nucleotides or polynucleotides including RNA or RNA-based or RNA derived API siRNA, micro RNA, antisense oligonucleotides, ribozymes, plasmids and/or immune stimulating nucleic acids.

It is well known in the art that LNPs with diameter 200 nm or smaller are able to better avoid removal by the reticuloendothelial system than larger sized particles resulting in circulating in a subject's bloodstream for a far longer period of time than larger sized LNPs. Furthermore, LNPs with diameter smaller than 100 nm are able to preferentially accumulate at disease sites such as tumors and sites of infection and inflammation due to their ability to extravasate through leaky vasculature in such regions due to the EPR effect as discussed in further detail below.

Therefore, in an embodiment, diameter of the LNP 1 of the present invention containing cDDP is from about 25 nm to about 200 nm such as about 25 nm, about 50 nm, about 75 nm, about 100 nm, about 125 nm, about 150 nm, about 175 nm or about 200 nm including all numbers ranges and numbers falling within these values. In an embodiment, diameter of the LNP 1 of the present invention containing dox is from about 25 nm to about 200 nm such as about 25 nm, about 50 nm, about 75 nm, about 100 nm, about 125 nm, about 150 nm, about 175 nm or about 200 nm including all numbers ranges and numbers falling within these values. In an embodiment, diameter of the LNP 1 of the present invention containing doxy is from about 25 nm to about 500 nm such as about 25 nm, about 50 nm, about 100 nm, about 150 nm, about 200 nm, about 250 nm, about 300 nm, about 350 nm, about 400 nm, about 450 nm or about 500 nm including all numbers ranges and numbers falling within these values. Because of the small diameters, some embodiments of the LNP 1 of the present invention may also be categorized as limit-size LNP.

It is also known in the art that charged particles are easily removed from blood circulation by the reticuloendothelial system. The LNP 1 of the present invention avoids this removal mechanism by having an overall near neutral or neutral electrostatic charge as the positive charge of the API 30 balances out with the negative charge of the anionic lipid 22. The overall charge of LNP 1 of the present invention is measured by dynamic light scattering using Nanoparticle analyzer SZ-100 (HORIBA, Kyoto, Japan). Therefore, in an embodiment, the overall charge of the LNP of the present invention may range from about ±100, ±75, ±60, ±50, ±40, ±30, ±20, ±10, ±5 or ±2 zeta value.

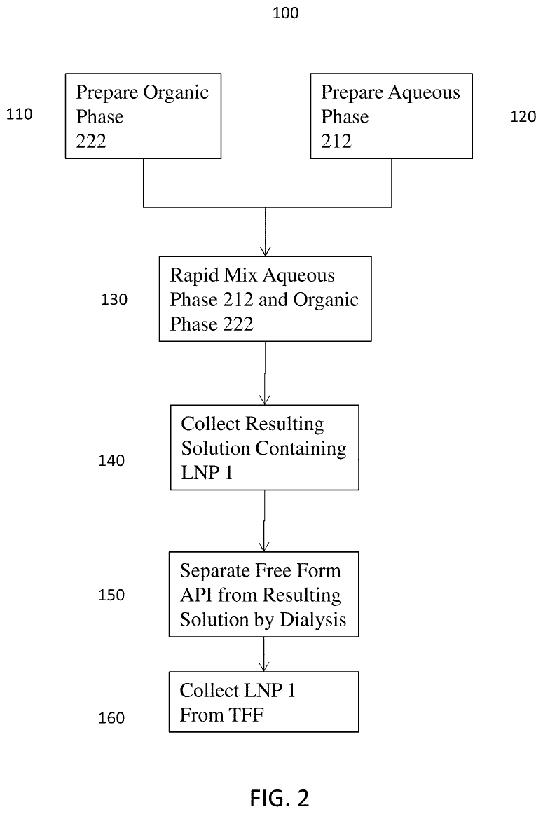

The present invention also comprises a method for preparing the LNP 1 of the present invention. An embodiment of the method of preparation 100 of the present invention is illustrated in FIG. 2. The method of preparation 100 of the present invention utilizes rapid mixing to amplify the effect of electrostatic attraction between charged components comprising the LNP 1 of the present invention to result in high DL ratio and high normalized release.

In an embodiment, the rapid mixing comprises mixing aqueous phase 212 and organic phase 222 wherein the organic phase 222 comprises both neutral lipids 12 as well as anionic lipids 22 dissolved in a solvent such as ethanol and the aqueous phase 212 comprises positively charged API 30 such as positively charged dox, doxy or cDDP dissolved in a solvent such as NaCl. In an embodiment, rapid mixing mixes anionic lipids 22 of organic phase 222 with cationic API 30 of aqueous phase 212 that quickly and efficiently disperse the anionic lipids 22 and cationic API 30 together to increase the effect of electrostatic attraction of the two components, raising DL ratio of the LNP 1 of the present invention. Rapid mixing can be distinguished from passive mixing. Specifically, whereas passive mixing comprises mixing that passively occurs by merely combining two or more fluids such as by injection of two or more fluids into a receptacle, rapid mixing comprises not only combining two or more fluids but also actively mixing two or more fluids with a mixing element 230 to quickly and uniformly disperse the fluids including their components. In another embodiment, rapid mixing comprises not only combining two or more fluids but also mixing the two or more fluids with a mixing element 230 in conjunction with pumping action of one or more fluid pumps 214 and 224 as discussed further below. The rapid mixing increases the effect of electrostatic attraction of the anionic lipids 22 and cationic API 30 to provide more efficient complexing of API 30 with anionic lipid 22 to result in the high D/L ratio and normalized release of the LNP 1 of the present invention. In an embodiment, the rapid mixing achieves uniform dispersion of the fluids and their components being mixed within about 100 microseconds to about 2 seconds such as about 100 microsecond, about 500 microsecond, about 1 milliseconds, about 50 milliseconds, about 100 milliseconds, about 200 milliseconds, about 500 milliseconds, about 800 milliseconds, about 1 second about 2 seconds including all numbers ranges and numbers falling within these values.

In an embodiment, cholesterol of the organic phase 222 can be replaced with nonamphiphilic fat such as medium chain triglyceride (MCT). In another embodiment, the solvent in which cholesterol is dissolved may be selected from methanol, isopropanol, and other water soluble organic solvents. In another embodiment, the solvent in which the positively charged API 30 is dissolved may be PBS, ddH₂O, sucrose or dextrose.

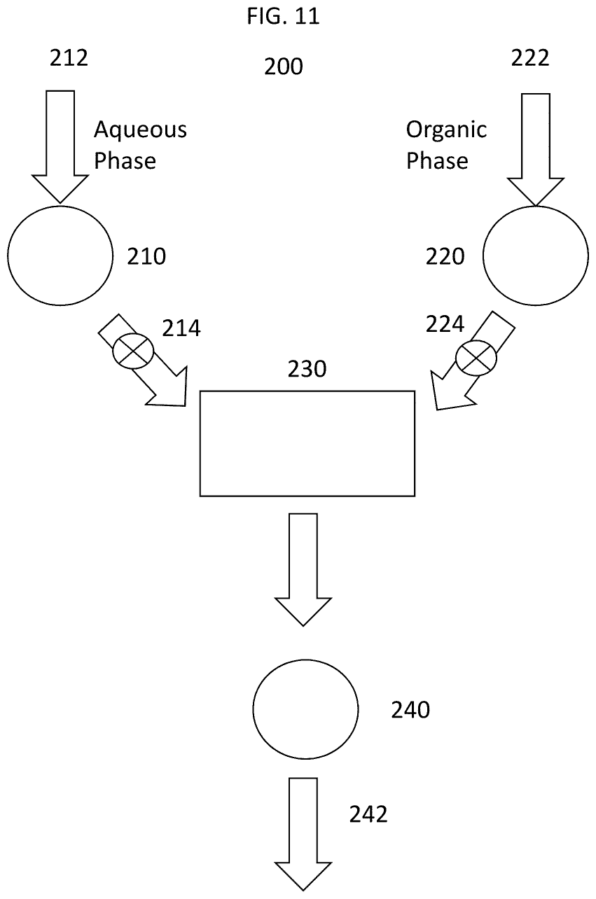

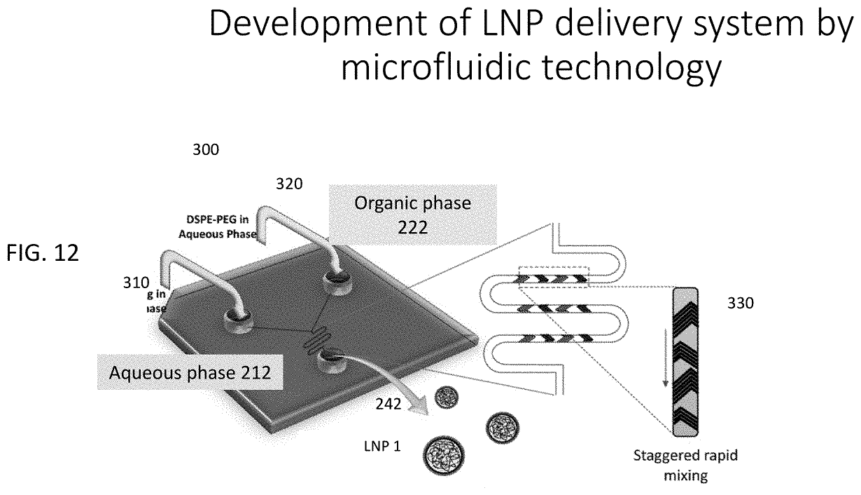

FIG. 11 illustrates an exemplary embodiment of rapid mixing system 200. As shown in FIG. 11, the rapid mixing system 200 comprises an aqueous phase receptacle 210, an aqueous phase pump 214, an organic phase receptacle 220, an organic phase pump 224, a mixing element 230 and resulting solution receptacle 240. The aqueous phase receptacle 210 is configured to receive the aqueous phase 212, and the organic phase receptacle 220 is configured to receive the organic phase 222. Aqueous phase pump 214 and organic phase pump 224 are each configured to pump the aqueous phase 212 and organic phase 224 to the mixing element 230 for rapid mixing, respectively. Preferably, the aqueous phase pump 214 and organic phase pump 224 may each be independently controlled to provide the desired flow volume and/or flow rate for each phase 212 and 222. In an embodiment, the mixing element 230 is configured to rapid mix the aqueous phase 212 and organic phase 222. In another embodiment, the mixing element 230 is configured to mix the aqueous phase 212 and organic phase 222 in conjunction with pumping action of the aqueous phase pump 214 and organic phase pump 224. In an embodiment, the mixing element 230 comprises mixing elements used in the art for rapid mixing fluids capable of achieving the desired result of quickly and effectively dispersing the anionic lipid 22 and positively charged API 30 uniformly to increase the effect of electrostatic attraction of those charged components. There are various mixing elements 230 capable of rapidly mixing anionic lipid 22 and positively charged API 30 in a way that substantially increases the effect of electrostatic attraction of those elements above and beyond what is possible by mere passive mixing. For example, in an embodiment, the mixing element 230 may be as simple as a T or Y junction or as complex as microfluidic devices with herringbone structure as shown in FIG. 12. Other mixing elements 230 that may be used herein include but are not limited to hydrodynamic flow focusing (HFF), staggered herringbone micromixer (SHM), bifurcating mixers, baffle mixers, T-junction mixing, etc. . . . The resulting solution receptacle 240 comprises a receptacle for receiving the resulting solution 242 resulting from rapid mixing by mixing element 230.

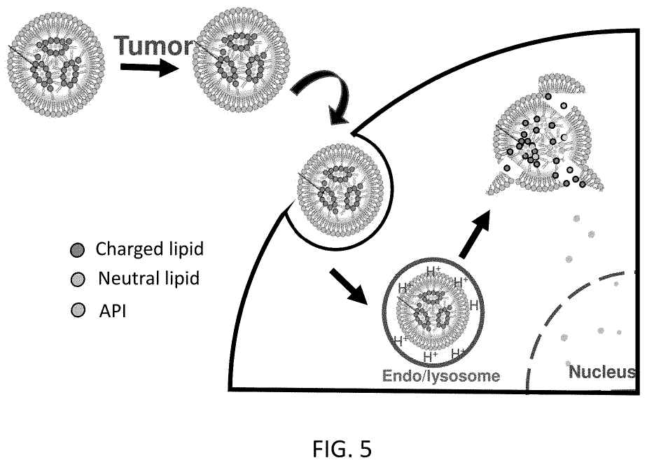

An exemplary rapid mixing system 200 comprises a microfluidic device 300 as shown in FIG. 11. In an embodiment, the rapid mixing with microfluidic device 300 with herringbone mixing element 330 may be adjusted by varying parameters such as flow rate ratio (FRR) and total flow rate (TFR) of the phases 212 and 222. FRR is defined as the ratio of flow rate of the organic phase 222 to the aqueous phase 212, and TFR is the sum of the two flow rates. These two parameters along with other factors such as lipid composition, ratio of charged lipid, cholesterol concentration, lipid concentration, drug loading, etc. . . . can influence characteristics of the resulting LNP 1 such as LNP diameter, D/L ratio, EE % etc. . . . discussed in further detail below in connection with FIG. 5. An embodiment of the microfluidic device 300 which is used in the Examples below is based off of the microfluidic device used in Belliveau et al.⁶, which features a mixing channel 200 μm wide and 79 μm high, with herringbone structures formed by 31 μm high and 50 μm thick features on the roof of the channel. Fluidic connections were made with 1/32″ I.D., 3/32″ O.D. tubing that was attached to 21G1 needles for connection with syringes. One mL or 3 mL syringes were used for inlet streams. Two syringe pumps were used to control the flow rate through the device. The syringe pump introduces the two solutions into the microfluidic device, where they come into contact at the Y-junction. ⁶ Belliveau, N. M. et al. (2012). Microfluidic Synthesis of Highly Potent Limit-size Lipid Nanoparticles for In Vivo Delivery of siRNA. Molecular Therapy—Nucleic Acids, 1, e37. https://doi.org/10.1038/mtna.2012.28

The rapid mixing may also be achieved using T or Y junction as mixing element 230. The flow rate for aqueous phase 212 may be from about 1 mL/min to about 6 mL/min, such as about 1 mL/min, about 2 mL/min, about 3 mL/min, about 4 mL/min, about 5 mL/min or about 6 mL/min including all numbers ranges and numbers falling within these values. The flow rate for organic phase 222 may be from about 2 mL/min to about 6 mL/min, such as about 2 mL/min, about 3 mL/min, about 4 mL/min, about 5 mL/min or about 6 mL/min including all numbers ranges and numbers falling within these values. The total flow rate may be from about 1 mL/min to 12 mL/min such as about 1 mL/min, about 2 mL/min, about 3 mL/min, about 4 mL/min, about 5 mL/min, about 6 mL/min, about 7 mL/min, about 8 mL/min, about 9 mL/min, about 10 mL/min, about 11 mL/min or about 12 mL/min including all numbers ranges and numbers falling within these values.

FIG. 2 illustrates an embodiment of the method of LNP preparation 100 of the present invention. As illustrated in FIG. 2, the first steps 110 and 120 involve preparation of organic phase 222 and preparation of aqueous phase 212, respectively. In an embodiment, the step 110 of preparation of the organic phase 222 comprises mixing the desired amount of neutral 12 as well as anionic lipids 22 in a solvent such as ethanol. In an embodiment, the organic phase 222 may further comprise cholesterol and/or PEG related components such as DSPE-PEG2000. In an embodiment, the step 120 of preparation of the aqueous phase solution 212 comprises mixing the desired amount of positively charged API 30 such as dox, cDDP or doxy in NaCl solution. In one embodiment, the drug concentration in the aqueous phase may range from about 1 mg/mL to about 20 mg/mL such as about 1 mg/mL, about 2 mg/mL, about 4 mg/mL, about 6 mg/mL, about 8 mg/mL, about 10 mg/mL, about 12 mg/mL, about 14 mg/mL, about 16 mg/mL, about 18 mg/mL or about 20 mg/mL including all numbers ranges and numbers falling within these values, and the lipid concentration in the organic phase may range from about 1 mg/mL to about 40 mg/mL such as about 1 mg/mL, about 3 mg/mL, about 5 mg/mL, about 10 mg/mL, about 15 mg/mL, about 20 mg/mL, about 25 mg/mL, about 30 mg/mL, about 35 mg/mL or about 40 mg/mL including all numbers ranges and numbers falling within these values. In addition, the reaction temperature for the preparation step ranged from room temperature of about 25° C. to about 65° C.

Next in step 130, the organic phase 222 and aqueous phase 212 undergo rapid mixing. As discussed above, rapid mixing comprises rapid mixing the organic and aqueous phases 222 and 212 with mixing element 230 to bring anionic lipid 22, neutral lipid 12 and charged API 30 into close contact with each other to facilitate formation of the LNP 1 of the present invention. In one embodiment, the rapid mixing step 130 is performed using a microfluidic device. In another embodiment, the rapid mixing step 130 is performed using a microfluidic device 300 with a herringbone structure. In yet another embodiment, the rapid mixing step 130 is performed using a microfluidic device 300 with a herringbone structure with TFR from about 10 to 14 ml/min and FRR from about 2:1 to 6:1. In another embodiment, the rapid mixing step 130 is performed using a T or a Y junction.

Next, in step 140, the resulting solution 242 is collected from resulting solution receptacle 240. Next, in step 150, LNP 1 of the present invention is isolated from the resulting solution 242 by dialysis such as by using a Tangential Filter Flow System (TFF). An embodiment of the TFF may be the mini MAP.03 System from LEF Science of Taipei, Taiwan. Finally, in step 160, LNP 1 of the present invention are collected from TFF and stored at about 4° C. away from light. After dialysis, the desired concentration can be achieved using TFF.

In an embodiment, the method of preparation 100 of the present invention comprises a one-staged process with respect to formation of LNP 1 of the present invention. Specifically, the lipid-API complexes 20 encapsulated by the outer lipid layer 10 are formed in one continuous process within step 130 as described above in connection with FIG. 2 rather than forming the lipid-API complexes 20 first in one process and then forming the outer lipid layer 10 around the lipid-API complexes 20 in a separate process. In an embodiment, the method of preparation 100 of the present invention provides all lipids necessary to form the LNP 1 of the present invention in one step 130 so that no further addition of lipids is necessary in any subsequent steps for the formation of LNP 1 of the present invention.

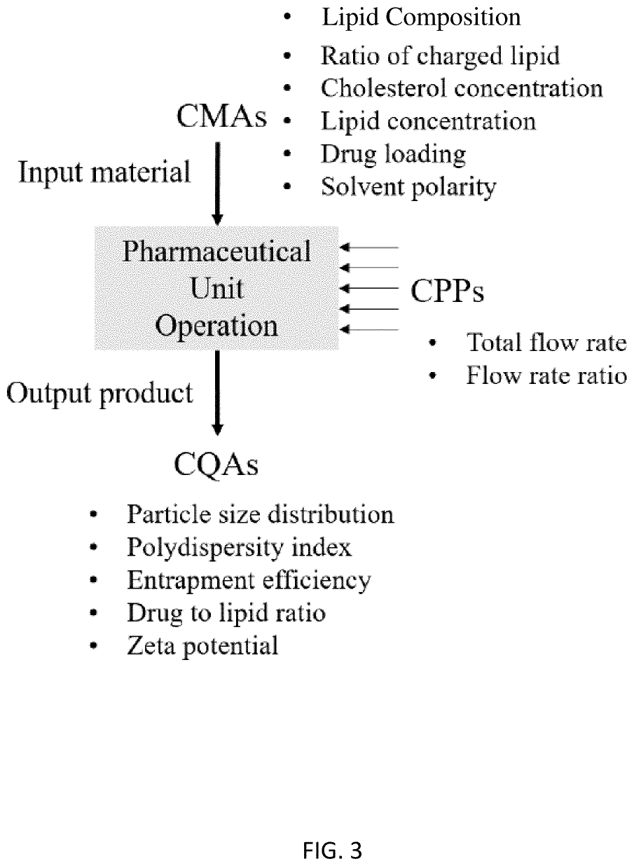

FIG. 3 illustrates various factors that can influence various characteristics of the LNP 1 of the present invention. As illustrated in FIG. 3, ratio of charged lipid to neutral lipid, cholesterol concentration, lipid concentration, drug loading, solvent polarity as well as lipid composition are some of the factors that can influence characteristics of the resulting LNP 1 of the present invention. These LNP characteristics include particle size distribution, polydispersity index, entrapment efficiency, drug to lipid ratio, zeta potential and/or normalized release.

In an embodiment, total lipids (weight/volume) is about 200 mg/mL to about 1 mg/mL such as about 200 mg/mL, about 180 mg/mL, about 160 mg/mL, about 140 mg/mL, about 120 mg/mL, about 100 mg/mL, about 80 mg/mL, about 70 mg/mL, about 60 mg/mL, about 50 mg/mL, about 40 mg/mL, about 30 mg/mL, about 20 mg/mL, about 10 mg/mL, about 5 mg/mL or about 1 mg/mL including all numbers ranges and numbers falling within these values. In an embodiment, the total lipid to cholesterol ratio ranges by weight may range from no cholesterol at all to 1:2 such as no cholesterol, about 10:1, about 7.5, about 5:1, about 2:1, about 1:1 or about 1:2 including all numbers ranges and numbers falling within these values. In an embodiment, anionic lipid to neutral lipid molar ratio is about 100:1 to about 0.1:1 such as about 100:1, about 90:1, about 80:1, about 70:1, about 60:1, about 50:1, about 40:1, about 30:1 about 20:1, about 15:1, about 7:1, about 5:1, about 3:1, about 0.75:1, about 0.5:1 or about 0.1:1 including all numbers ranges and numbers falling within these values. In an embodiment, drug loading is about 100 mg/mL to about 0.1 mg/mL such as about 100 mg/mL, about 90 mg/mL, about 80 mg/mL, about 70 mg/mL, about 60 mg/mL, about 50 mg/mL, about 40 mg/mL, about 30 mg/mL, about 20 mg/mL, about 10 mg/mL, about 8 mg/mL, about 6 mg/mL, about 4 mg/mL, about 2 mg/mL, about 1 mg/mL or about 0.1 mg/mL including all numbers ranges and numbers falling within these values. In an embodiment, TFR used in the microfluidic device 300 is about 30 to about 0.1 ml/min such as about 30 ml/min, about 25 ml/min, about 20 ml/min, about 15 ml/min, about 10 ml/min, about 5 ml/min, about 2 ml/min, about 1 ml/min or about 0.1 ml/min including all numbers ranges and numbers falling within these values. In an embodiment, the FRR used in microfluidic device 300 ranges between about 10:1 to about 1:1 such as about 10:1, about 8:1, about 6:1, about 4:1, about 2:1 or about 1:1 including all numbers ranges and numbers falling within these values. In an embodiment, the organic phase may comprise both ethanol (EtOH) and isopropanol. In another embodiment, the organic phase may comprise organic solutions such as methanol, EtOH, isopropanol, or other organic solutions which can dissolve in aqueous phase.

In an embodiment, the entrapment efficiency of lipo-doxy is greater than about 20%, greater than about 30%, greater than about 40%, greater than about 50%, greater than about 60% or about 65%.

In an embodiment, the polydispersity index of the LNP 1 of the present invention is lower than 0.5, lower than 0.3, lower than 0.2 or about 0.165.

In an embodiment, the normalized ratio for LNP1 of the present invention is greater than 1.2, greater than 1.3, or greater than 1.4.

In an embodiment, the LNP 1 of the present invention comprises anionic EPG, neutral HSPG, DSPE-mPEG2000, 14:0 PEG 2000 PE lipids or a combination thereof. In an embodiment, the organic phase 222 comprises molar ratio of anionic lipid 22 to neutral lipid 12 at about 15 to 1 such as about 15, about 13, about 11, about 9, about 7, about 5, about 3, about 2 or about 1 including all numbers ranges and numbers falling within these values. In another embodiment, the LNP 1 of the present invention comprises anionic EPG and neutral HSPG lipids at molar ratio of about 10 to about 2.5 such as about 10, about 9, about 8, about 7, about 6, about 5, about 4, about 3 or about 2.5 including all numbers ranges and numbers falling within these values. In yet another embodiment, the LNP 1 of the present invention further comprises DSPE-mPEG2000 at about 0-4 molar ratio and/or 14:0 PEG 2000 PE at about 0:4 molar ratio. In an embodiment, the organic phase 222 comprises EPG at about 25 to 65 molar ratio such about 25, about 30, about 35, about 40, about 45, about 50, about 55, about 60 or about 65 including all numbers ranges and numbers falling within these values, HSPG at about 5-15 molar ratio such about 5, about 6, about 7, about 8, about 9, about 10, about 11 about 12, about 13 about 14 or about 15 including all numbers ranges and numbers falling within these values. In an embodiment, the molar ratio of anionic lipids 22 to neutral lipids 12 in the LNP 1 of the present invention is the same as the molar ratio of organic phase 222.

The present invention also comprises method of treatment of illnesses such as cancer using any embodiments of the LNP 1 of the present invention disclosed comprising administration of the LNP 1 of the present invention to the subject. In an embodiment, the present invention is a method of treatment of illnesses treatable by dox, doxy or cisplatin.

According to the invention, the administration can be rectal, nasal, vaginal, parenteral or topical. More preferably, the administration is parenteral including but not limited to intravenous, subcutaneous, intramuscular, intradermal and intraperitoneal. More preferably, the administration is intravenous. Preferably, the targets are tumors. Preferably, the API 30 comprises an anti-tumor drug or an antibiotic.

According to the invention, after administering LNP 1 of the present invention to a subject, the LNPs accumulate at the tumor sites due to enhanced permeability and retention effect (EPR). Specifically, EPR results from the fact that tumors tend to be nutrient and oxygen rich due to fast growing blood vessels in tumors. However, those fast growing blood vessels comprise irregularly positioned endothelia cells resulting in gaps that allow nanoparticles of 200 nm or less such as the LNP 1 of the present invention to escape blood vessels. Combining this with the fact that tumors lack lymphatic systems that can flush away nanoparticles, EPR effects result in LNPs 1 of the present invention accumulating at the tumor.

As illustrated in FIG. 5, LNPs 1 of the present invention may enter lysosomes of the tumor cells via endocytosis. This environment is hydrogen ion rich as illustrated in FIG. 5. These positively charged hydrogen ions result in electrostatic imbalance for charged components such as charged lipid 22 and charged API 30 of the lipid-API complexes 20 that causes the lipid-API complexes 20 to break apart which, in turn, causes the LNP monolayer 10 to also break apart, releasing the API into tumor cells. In this manner, the LNP 1 of the present invention substantially improves API release and therapeutic effects as shown in the Examples below.

We claim the therapy of subject, e.g., mammals such as mice, rats, simians, and human patients, with cancers including, but not limited to breast, prostate, colon, non-small lung, pancreatic, testicular, ovarian, cervical carcinomas, head and neck squamous cell carcinomas.

As used herein, “administration, delivered or administered” is intended to include any method which ultimately provides the drug/LNP complex to the tumor mass. Examples include, but are not limited to, topical application, intravenous administration, parenteral administration or by subcutaneous injection around the tumor. Tumor measurements to determine reduction of tumor size are made in two dimensions using vernier calipers twice a week.

For in vivo administration, the pharmaceutical compositions are preferably administered parenterally, i.e., intravenously, intraperitoneally, subcutaneously, intrathecally, injection to the spinal cord, intramuscularly, intraarticularly, portal vein injection, or intratumorally. More preferably, the pharmaceutical compositions are administered intravenously or intratumorally by a bolus injection.

Administration in vivo can be effected in one dose, continuously or intermittently throughout the course of treatment. Methods of determining the most effective means and dosage of administration are well known to those of skill in the art and will vary with the composition used for therapy, the purpose of the therapy, the target cell being treated, and the subject being treated. Single or multiple administrations can be carried out with the dose level and pattern being selected by the treating physician. Suitable dosage formulations and methods of administering the agents can be empirically determined by those of skill in the art.

It should be noted that all preferred embodiments discussed for one or several aspects of the invention also relate to all other aspects. This particularly refers to the amount and type of neutral and/or anionic lipid, the amount and type of rapid agent, the amount and type of further rapid agent for combination therapy, and the type of disorder to be treated.

The following examples should be illustrative only but are not meant to be limiting to the scope of the invention. Other generic and specific configurations will be apparent to those skilled in the art.

### EXAMPLES

LNP 1 of the present invention of Examples 1-2 were prepared using the method of preparation 100 of the present invention. The organic phase 222 for preparing the LNP 1 of the present invention comprises EPG at about 46.8% molar ratio, HPSC at about 9.4% molar ratio, cholesterol at about 41.9% molar ratio and DSPE-mPEG2000 at about 1.9% molar ratio dissolved in EtOH at total lipid concentration about 18.6 mg/mL. The aqueous phase 212 for preparing the LNP 1 of the present invention comprise Dox in NaCl at concentration of about 0.9%. Dox was initially loaded at from about 1 mg/mL to about 8 mg/mL. Dox may also be loaded at any concentration range at which dissolution can be achieved. The organic and aqueous phases are then rapidly mixed using microfluidic device (Nanoassembler benchtop) with herringbone structure using flow parameters TFR about 12 mL/min and FRR about 4:1. The resulting LNP 1 of the present invention has diameter about 75.6, PDI about 0.174, DL ratio of about 0.44 and encapsulation efficiency (EE) about 66%. The experiments were carried out at room temperature of about 25° C.

LNP doxy 1 of the present invention of Example 3 was made in the same way as LNP dox 1 of the present invention of Examples 1 and 2, with the only difference being the API.

The Examples use the following method to determine concentration of doxorubicin: First, 10 mg/mL dox was diluted in 95% methanol to various concentrations at 10, 5, 2.5, 1.25, 0.625, 0.3125 g/mL to serve as calibration solutions. The dox sample in question requiring concentration measurement is diluted with 95% methanol to 100 times by volume. Then, UV-Visible spectrophotometer at 470 nm wavelength light was used to determine dox concentration by interpolation after comparison with the calibrating solutions. All processes conducted at room temperature of about 25° C.

### Example 1—In Vitro Release Drug Release of LNPs of the Present Invention Encapsulating Dox in Acidic Environments

During experiments, PBS buffer was used to simulate a physiological environment at pH of 7.4, while citrate buffer was used to simulate a tumor microenvironment at pH of 5.4, wherein the citrate buffer contains sodium citrate dehydrate and citric acid and its pH can be adjusted using NaOH and HCl. The in vitro API release was evaluated in these two different solutions using the following steps:

1 mg/mL LNP-dox solution in PBS buffer was prepared by diluting LNP-dox using PBS buffer. The solution was placed into a 1 mL/5 cm dialysis tube (Float-A-lyzer), in which the pore size is 100 kDa. The tube was placed into a beaker containing 150 mL dialysis buffer. The beaker was then placed into a 37° C. water bath. Samples of 1 mL solution were taken from the beaker at 0, 0.5, 1, 2, 4, 8, 26, and 31 hour(s). The API content in the samples was analyzed using UV-Vis spectrophotometer such as the Spectra/Por float-A-lyer G2. Following each sampling process, 1 mL of dialysis solution from the tube was added to the beaker to maintain consistent volume. Three forms of the API dox were used, including free form dox, LNP 1 encapsulating dox of the present invention, and Lipo-dox (Doxil®). It should be noted that this dialysis process is to determine the amount of API 30 released and has nothing to do with preparation or formation of the LNP 1 of the present invention.

1 mg/mL LNP-dox solution in citrate buffer was prepared by performing the aforementioned steps but replacing PBS buffer with citrate buffer.

FIG. 6 illustrates results of the experiment. As shown in FIG. 6, free form dox released the fastest of the three as expected. The LNP 1 of the present invention exhibited substantially faster release rate than Lipo-dox at both pH levels, with release rate at acidic environment of pH 5.4 being the highest, approximating the release rate of free form dox at 31 hours and about 40% more released than compared to pH 7.4 environment. The substantially faster release rate in acidic environment as compared to the pH 7.4 environment demonstrates that the LNP 1 of the present invention has substantially higher normalized release of about 1.4 than Lipo-dox and free form Dox which have normalized release of about 1, indicating the LNP 1 of the present invention is far more capable of targeting diseased sites such as tumors or areas of inflammation which have an acidic environment than both free form dox and Lipo-dox.

### Example 2—In Vivo Therapeutic Efficacy of LNP of the Present Invention Encapsulating Dox

C26 human cancer cell line was implanted into the backs of BALB/c mice to create colon-26 carcinoma tumor-bearing mice. When the tumor grew to 100 mm³, LNP 1 of the present invention containing dox was injected via mice's vein located in the tail at a dosage of 5 mg/kg of dox, once per week. At predetermined time periods 1, 2, 5, 7, 9, 12, 14, 19, 21, 22 days, tumor volume was determined by Vernier caliper. The experiment continued until either mouse death or the tumor size exceeding 2,500 mm³.

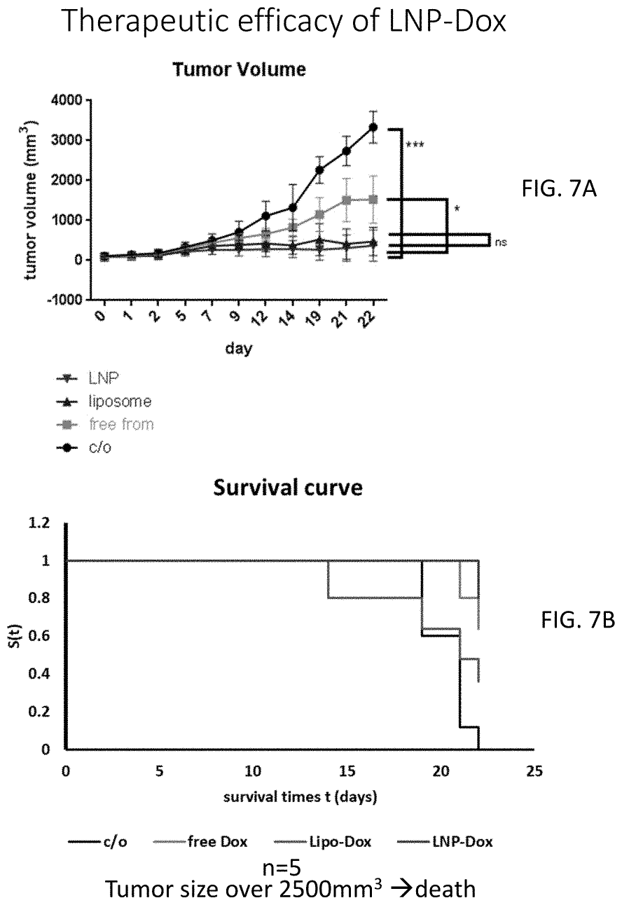

FIG. 7 illustrates results of the experiment. As shown in FIG. 7, both Lipo-dox and the LNP 1 of the present invention resulted in substantially lower tumor volume than control and free form dox up to 22 days of the experiment. With regards to the survival curve, the LNP 1 of the present invention only resulted in 20% death or has tumor size of over 2500 mm³ at the end of the experiment. In comparison, 60% of lipo-dox treated mice died or had tumor size of over 2500 mm³. 40% of mice treated with free dox died or had tumor size of over 2500 mm³. Finally, 100% of the mice in control group died or had tumor size of over 2500 mm³. The trials demonstrate that LNP of the present invention provide substantially superior therapeutic effects than Lipo-dox and free form dox while providing better protection than free form dox from toxicity.

### Example 3—Antibacterial Susceptibility of LNP-Doxycycline

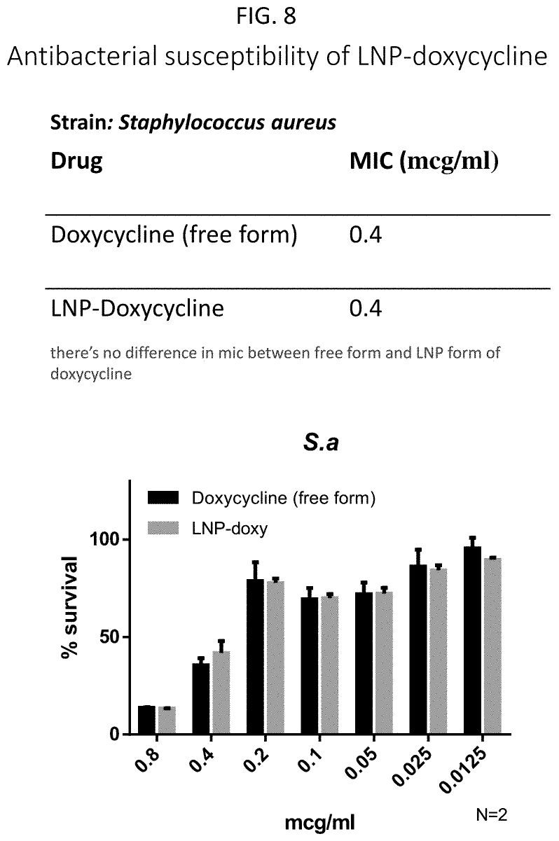

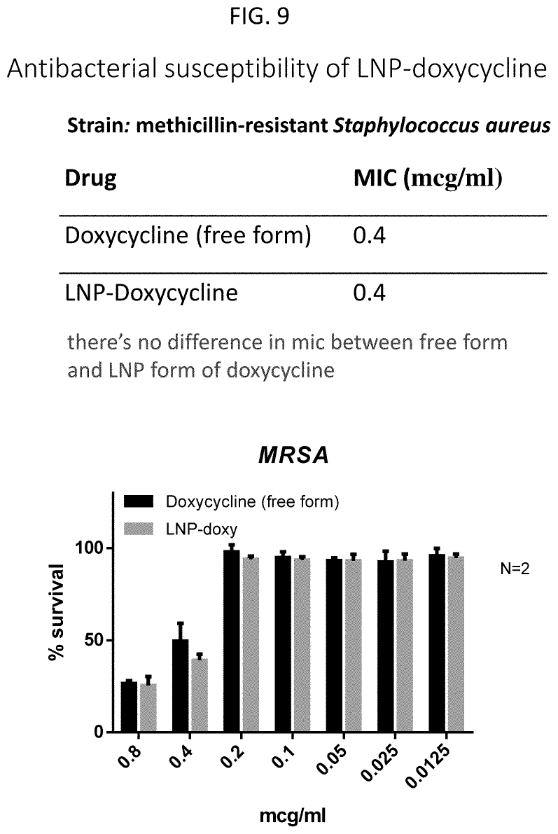

Free form doxycycline and LNP 1 of the present invention containing doxy were separately applied to Staphylococcus aureus bacteria as well as methicillin-resistant Staphylococcus aureus at 10⁵ CFU, incubation with LNP for about 24 hour at about 37° C. As illustrated in FIGS. 8 and 9, therapeutic effect of free form and LNP doxycycline are similar at various dosages.

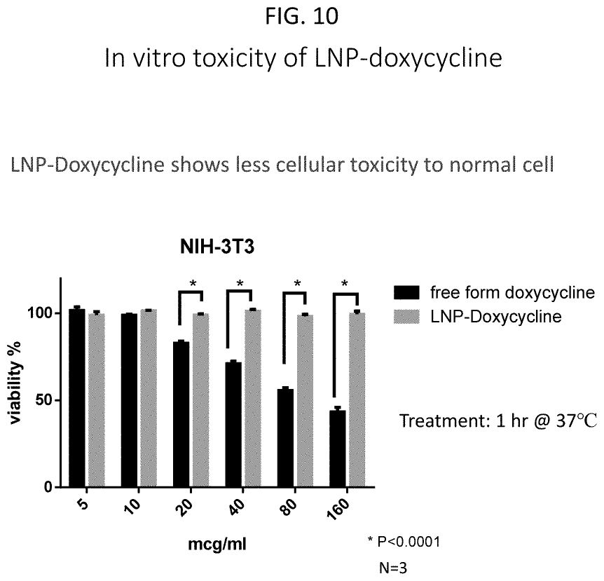

Free form doxycycline and LNP 1 of the present invention were separately applied to NIH-3T3 cells cultured in high glucose Dulbecco's Modified Eagle's Medium containing 10% FBS and 1% penicillin/streptomycin. The cell lines were cultured at 37° C. in a humidified incubator with 5% CO2/50,000 cell/mL for 1 hour. As shown in FIG. 10, LNP-doxy 1 of the present invention is clearly less toxic than free form doxycycline to NIH-3T3 cells and therefore less toxic to normal human cells.

1-28. (canceled)

29. A lipid-based nanoparticle (LNP) comprising an outer lipid monolayer encapsulating a plurality of lipid-active pharmaceutical ingredient (API) complexes,
wherein each lipid-API complex comprises a complex of anionic lipid and API;
wherein the API comprises a positively charged form of an API; and
wherein the outer lipid monolayer of the LNP comprises neutral lipids.

30. The LNP of claim 29, wherein the molar ratio of anionic to neutral lipid is from about 12 to about 2.5.

31. The LNP of claim 30, wherein the anionic lipid comprises egg L-α-phosphatidylglycerol (EPG) and the neutral lipid comprises L-α-phosphatidylcholine, hydrogenated soybean phosphatidylcholine (HSPC).

32. The LNP of claim 30, wherein the LNP further comprises cholesterol at ratio by weight of total lipid to cholesterol ranging from about 10:1 to about 1:2.

33. The LNP of claim 29, wherein the API is a positively charged form of doxorubicin or a pharmaceutically acceptable salt form thereof.

34. The LNP of claim 33, wherein the drug to lipid ratio is greater than about 0.15.

35. The LNP of claim 33, wherein the over 80% of doxorubicin is released in about 31 hours in a pH 5.4 citrate buffer environment at about 37° C.

36. The LNP of claim 33, wherein the normalized release is greater than about 1.2, about 1.3 or about 1.4.

37. The LNP of claim 29, wherein the positively charged API is a positively charged form of doxycycline or a pharmaceutically acceptable salt form thereof.

38. The LNP of claim 37, wherein the drug to lipid ratio is greater than about 0.16.

39. The LNP of claim 29, wherein the positively charged antineoplastic drug is a positively charged form of cisplatin.

40. The LNP of claim 39, wherein the drug to lipid ratio is greater than about 0.12.

41. The LNP of claim 29 is made using rapid mixing.

42. The LNP of claim 41, wherein the rapid mixing is performed with microfluidic device with herringbone mixing element.

43. The LNP of claim 29, wherein the LNP does not comprise a therapeutically effective amount of nucleic acids, nucleotides or polynucleotides.

44. The LNP of claim 29, wherein the anionic lipid does not comprise any therapeutically effective amount of phosphatidyl glycerol or phosphatidyl glycerol derivative.

45. A method of preparation for the LNP of claim 29 comprising the steps of
a. Prepare organic phase fluid comprising neutral and anionic lipids.
b. Prepare aqueous phase fluid comprising a positively charged form of an active pharmaceutical ingredient (API).
c. Rapid mix the organic and the aqueous phases to make a resulting solution.
d. Isolate the LNP from the resulting solution using dialysis process.

46. The method of preparation of claim 45, wherein the API comprises a positively charged form of doxorubicin, doxycycline, cisplatin or a pharmaceutically acceptable salt form thereof.

47. The method of preparation of claim 45, wherein the rapid mixing step is performed using a rapid mixing system comprising an organic phase pump, an aqueous phase pump and a mixing element.

48. The method of preparation of claim 47, wherein the mixing element is a T junction or a Y junction.

49. The method of preparation of claim 47, wherein the mixing element is a microfluidic device comprising herringbone mixing structure.

50. The method of preparation of claim 45, wherein the molar ratio of anionic to neutral lipid is from about 12 to about 2.5

51. The method of preparation of claim 50, wherein the anionic lipid comprises egg L-α-phosphatidylglycerol (EPG) and neutral lipid comprises L-α-phosphatidylcholine, hydrogenated soybean phosphatidylcholine (HSPC).

52. The method of preparation of claim 46, wherein the drug to lipid ratio of the LNP encapsulating doxorubicin or a pharmaceutically acceptable salt form thereof is greater than about 0.15.

53. The method of preparation of claim 46, wherein drug to lipid ratio of the LNP encapsulating doxycycline or a pharmaceutically acceptable salt form thereof is greater than about 0.16.

54. The method of preparation of claim 46, wherein drug to lipid ratio of the LNP encapsulating cisplatin or a pharmaceutically acceptable salt form thereof is greater than about 0.12.

55. The method of claim 45, wherein the LNP does not comprise a therapeutically effective amount of nucleic acids, nucleotides or polynucleotides.

56. A method of treatment using the LNP of claim 29 comprising the step of injecting the LNP of claim 1 into a subject at dosage of about 0.01 mg/mL to 50 mg/mL.
